(19) United States
(12) Patent Application Publication
(10) Pub. No.: US 2010/0047483 A1
(43) Pub. Date: Feb. 25, 2010

(54) BIREFRINGENT FILM, LAMINATED FILM, AND IMAGE DISPLAY DEVICE

(75) Inventor: Shoichi Matsuda, Ibaraki-shi (JP)

Correspondence Address: WESTERMAN, HATTORI, DANIELS & ADRIAN, LLP, 1250 CONNECTICUT AVENUE, NW, SUITE 700, WASHINGTON, DC, 20036, US

(73) Assignee: NITTO DENKO CORPORATION, Ibaraki-shi, Osaka (JP)

(21) Appl. No.: 12/594,889

(22) PCT Filed: Mar. 3, 2008
(86) PCT No.: PCT/JP2008/053747

§ 371 Date: Oct. 6, 2009

(30) Foreign Application Priority Data
Apr. 9, 2007 (JP) 2007-101256

(51) Int. Cl. C09K 19/34; C07D 241/36

(52) U.S. Cl. 428/1.3; 544/342

(57) ABSTRACT

The present invention provides a birefringent film having an index ellipsoid satisfying a relationship of nz>nx>ny, and having thinness and light weight, which can be relatively easily produced.

The birefringent film of the present invention contains an acenaphtho[1,2-b]quinoxaline derivative represented by the following general formula (I), and an index ellipsoid of the film satisfies a relationship of nz>nx>ny.

Here, in the formula (I), X, Y, and Z each independently represent a halogen atom, an alkoxy group, an —R group, an —OM group, a —COOM group, an —OCOR group, an —NHCOR group, a —CONHR group, an —NH₂ group, an —NO₂ group, a —CF₃ group, a —CN group, an —OCN group, an —SCN group, an —SM group, and a —PO₃M group; M represents a counterion; R represents hydrocarbon; k, l, m, and n represent the number of substitutions, k is an integer of 1 to 4, and l, m and n are each independently an integer of 0 to 3.

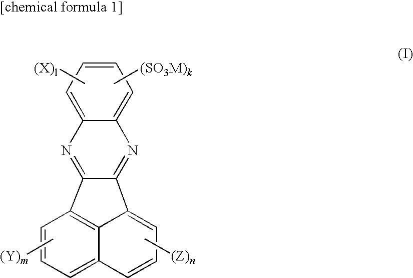

### TECHNICAL FIELD

The present invention relates to a birefringent film which is suitable as a constituent member of an image display device, as well as to a laminated film and an image display device having the birefringent film.

### BACKGROUND ART

A liquid crystal display is one of a device for displaying characters and images by utilizing electro-optical properties of liquid crystal molecules. However, the liquid crystal display utilizes liquid crystal molecules having optical anisotropy, so that excellent display properties are exhibited in one direction, while a screen becomes dark or unclear in other directions. Therefore, a birefringent film (also refers to a retardation film, an optical compensation layer, and the like) is provided with the liquid crystal display.

Typically, a birefringent film such that an index ellipsoid satisfies a relationship of nz>nx>ny is known as one of birefringent films (Patent Document 1). The birefringent film satisfying such a relationship of refractive index can be generally produced by attaching a shrinkable film to both sides of a polymeric film, and drawing the resulting polymeric film in such a manner as to expand in the thickness direction.

### Patent Document 1: Japanese Unexamined Patent Application Publication No. 2000-231014

### DISCLOSURE OF THE INVENTION

However, the birefringent film composed of a polymeric film thus prepared is likely to increase in the thickness. Thus, a liquid crystal display including the birefringent film becomes relatively thick and heavy. Therefore, the liquid crystal display cannot respond to requirement to reduce the thickness and the weight thereof.

Further, in producing the above birefringent film, a treatment of drawing the polymeric film in the thickness direction by utilizing a shrinking force of the shrinkable film is required.

It is an object of the present invention to provide a birefringent film having an index ellipsoid satisfying a relationship of nz>nx>ny and having thinness and light weight, and furthermore to provide a birefringent film which can be relatively easily produced.

Further, it is another object of the present invention to provide a laminated film and an image display device having the above-mentioned birefringent film.

A birefringent film of the present invention is characterized in that the birefringent film contains a lyotropic liquid crystalline polycyclic compound having an —SO₃M group (M represents a counterion), and has an index ellipsoid satisfying a relationship of nz>nx>ny.

Preferably, the above-mentioned polycyclic compound is an acenaphtho[1,2-b]quinoxaline derivative represented by the following general formula (I).

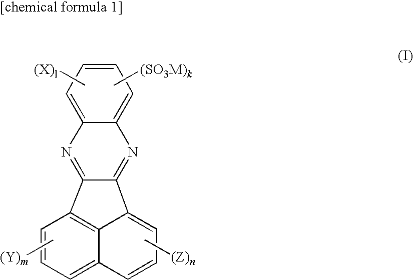

Here, in the formula (I), X, Y, and Z each independently represent a halogen atom, an alkoxy group, an —R group, an —OM group, a —COOM group, an —OCOR group, an —NHCOR group, a —CONHR group, an —NH₂ group, an —NO₂ group, a —CF₃ group, a —CN group, an —OCN group, an —SCN group, an —SM group, or a —PO₃M group (M represents a counterion and R represents hydrocarbon). The k, l, m, and n represent the number of substitutions, the k is an integer of 1 to 4, and the l, m, and n are each independently an integer of 0 to 3.

The birefringent film can be formed, for example, by applying a solution containing the above-mentioned lyotropic liquid crystalline polycyclic compound as a main component. Accordingly, the birefringent film of the present invention can be formed thin and the production thereof is relatively easily performed.

Further, since the birefringent film contains a polycyclic compound having an —SO₃M group, it becomes a film having an index ellipsoid satisfying a relationship of nz>nx>ny.

The laminated film of the present invention is characterized in that the birefringent film is laminated on another film.

Also, the image display device of the present invention is characterized in that the birefringent film or the laminated film is provided with the image display device.

The image display device including the birefringent film of the present invention is superior in thinness and light weight and is also superior in viewing angle characteristics.

The birefringent film of the present invention is useful as an optical compensation material of an image display device since the index ellipsoid thereof satisfies a relationship of nz>nx>ny. Furthermore, since the birefringent film of the present invention can be formed in a small thickness, the image display device including the birefringent film is superior in thinness and light weight.

### BEST MODE FOR CARRYING OUT THE INVENTION

### Term Meaning of the Present Invention

In the present invention, meanings of main terms are as follows.

“Birefringent film” means a film exhibiting a birefringence (anisotropy of refractive index) in the plane direction and/or the thickness direction. The “birefringent film” includes, for example, a film exhibiting a birefringence index in the plane direction and/or the thickness direction at a wavelength of 590 nm of 1×10⁻⁴ or more.

Also, “nx” and “ny” mean refractive indexes in the direction orthogonal to each other in the plane of a birefringent film (Here, nx>ny).

Further, “nz” means a refractive index in the thickness direction of a birefringent film.

“Transmittance (T[λ])” means the transmittance of light rays in visible light (typically a wavelength of 590 nm). The transmittance refers to a Y value obtained by making a luminous correction to spectral data measured at a film thickness of 100 μm with a spectrophotometer.

“In-plane birefringence index (Δn_{xy}[λ])” means difference in refractive index in the plane of a birefringent film measured at 23° C. with a wavelength of λ(nm). The Δn_{xy}[λ] can be determined by Δn_{xy}[λ]=nx−ny.

“In-plane retardation value (Re[λ])” means retardation value in the plane of a birefringent film measured at 23° C. with a wavelength of λ(nm). The Re[λ] can be determined by Rth[λ]=(nx−nz)×d when the thickness of a birefringent film is regarded as d (nm).

“Retardation value in the thickness direction (Rth[λ])” means a retardation value in the thickness direction of a birefringent film measured at 23° C. with a wavelength of λ(nm). The Rth[λ] can be determined by Rth[λ]=(nx−nz)×d when the thickness of a birefringent film is regarded as d (nm).

“Nz coefficient” means a value calculated by Rth[λ]/Re[λ]. In the present invention, the Nz coefficient is a value calculated by Rth[590]/Re[590] based on a wavelength of 590 nm. The meanings of Rth[590] and Re[590] are as described above.

Here, these values can be measured by the method described in the following Examples.

“Lyotropic liquid crystalline property” means a property to generate phase transition between an isotropic phase and a liquid crystal phase by changing a temperature or a concentration of a compound (a solute). The liquid crystal phase can be confirmed by optical patterns of the solution observed by a polarization microscope.

### <Birefringent Film of the Present Invention>

A birefringent film of the present invention contains a lyotropic liquid crystalline polycyclic compound having an —SO₃M group (M represents a counterion), and has an index ellipsoid satisfying a relationship of nz>nx>ny. The lyotropic liquid crystalline polycyclic compound is a compound capable of exhibiting a liquid crystal phase in a solution state. This liquid crystal phase is not particularly limited, and can be a nematic liquid crystal phase, a smectic liquid crystal phase, or a cholesteric liquid crystal phase. Preferably the liquid crystal phase is a nematic liquid crystal phase.

The polycyclic compound is preferably an acenaphtho[1,2-b]quinoxaline derivative having an —SO₃M group and more preferably an acenaphtho[1,2-b]quinoxaline derivative represented by the following general formula (I).

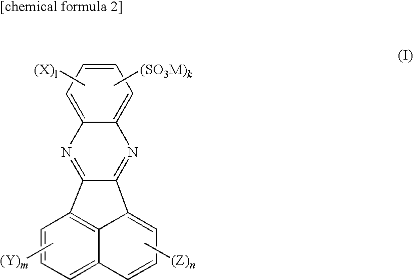

Here, in the formula (I), X, Y, and Z each independently represent a halogen atom, an alkoxy group, an —R group, an —OM group, a —COOM group, an —OCOR group, an —NHCOR group, a —CONHR group, an —NH₂ group, an —NO₂ group, a —CF₃ group, a —CN group, an —OCN group, an —SCN group, an —SM group, or a —PO₃M group (M represents a counterion, and R represents hydrocarbon). The k, l, m, and n represent the number of substitutions. The k is an integer of 1 to 4, and the l, m and n are each independently an integer of 0 to 3.

The M is preferably a hydrogen ion, an alkali metal ion, an alkaline earth metal ion, the other metal ion, or a substituted or unsubstituted ammonium ion. This metal ion includes, for example, Ni²⁺, Fe³⁺, Cu²⁺, Ag⁺, Zn²⁺, Al³⁺, Pd²⁺, Cd²⁺, Sn²⁺, Co²⁺, Mn²⁺, Ce³⁺, or the like. For example, when a birefringent film is formed from a solution containing the above-mentioned polycyclic compound, in the polycyclic compound, its substituent, M, is preferably an ion which improves the solubility in water. The polycyclic compound can be used for preparing a good aqueous solution since it is easily dissolved in water. After the birefringent film is formed by using this aqueous solution, the above-described ion which improves the solubility in water may be replaced with an ion which is insoluble in water or is difficult to dissolve in water in order to enhance the water resistance of the birefringent film.

In the formula (I), the hydrocarbon represented by the R is not particularly limited, and includes an optionally-substituted alkyl group (preferably an alkyl group having a carbon number of 1 to 6), an optionally-substituted cyclic alkyl group (preferably a cyclic alkyl group having a carbon number of 3 to 6), an optionally-substituted alkenyl group (preferably an alkenyl group having a carbon number of 2 to 6), an optionally-substituted aryl group (preferably an aryl group having one benzene ring), and the like. In the formula (I), the alkoxy group preferably has a carbon number of 1 to 6.

In the formula (I), X, Y, and Z are each independently an —OM group, a —COOM group, an —NH₂ group, an —SM group, or a —PO₃M group. Also, substitution numbers l, m, and n are preferably from 0 to 2 and more preferably from 0 to 1. These polycyclic compounds are excellent in solubility in a water-based solvent.

The birefringent film of the present invention can be obtained by applying a solution containing the above polycyclic compound onto an appropriate substrate to form a coated film. The coated film (birefringent film) has an index ellipsoid satisfying a relationship of nz>nx>ny. An action by which the index ellipsoid of the birefringent film containing the above polycyclic compound has a relationship of nz>nx>ny is not definite, but it is assumed as follows. That is, it is assumed that when the polycyclic compound is applied onto a substrate, a liquid crystal phase is formed in a state where an —SO₃M group in a molecule of the polycyclic compound is oriented to face the surface of the substrate. Therefore, the polycyclic compound is oriented in a state where the longitudinal direction of the polycyclic compound is substantially parallel to the normal direction to the surface of the substrate. By orienting the polycyclic compound in this way, a birefringent film in which the nz (a refractive index in the thickness direction) reaches the highest value can be obtained.

In consideration of the above-mentioned action, the polycyclic compound is preferably a compound having an —SO₃M group at an end of the longitudinal direction of a basic skeleton. These polycyclic compounds are, for example, acenaphtho[1,2-b]quinoxaline derivatives represented by the following general formulae (II) and (III).

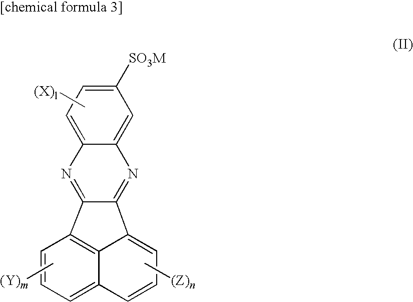

Here, in the formula (II), M, X, Y, and Z are the same as the above formula (I). The l, m, and n represent the number of substitutions, and each independently represent an integer of 0 to 3.

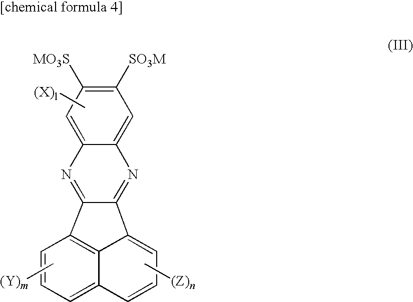

Here, in the formula (III), M, X, Y, and Z are the same as the above formula (I). The l, m, and n represent the number of substitutions. The m and n each independently are an integer of 0 to 3 and the l is an integer of 0 to 2.

Among the polycyclic compounds represented by the formula (II) or the formula (III), at least one of the l, m, and n is 0 (namely, at least one of the X, Y, and Z is not substituted) and more preferably all of the l, m, and n are 0 (namely, all of the X, Y, and Z are not substituted). Such a preferable polycyclic compound has less interaction of a substituent other than the —SO₃M group and is likely to be oriented such that the longitudinal direction of the polycyclic compound is substantially orthogonal to the surface of the substrate. Therefore, in the birefringent film containing the preferable polycyclic compound, the index ellipsoid can satisfy a relationship of nz>nx>ny with certainty. Further, the polycyclic compound is also superior in solubility in a water-based solvent.

Particularly preferably, all of the X, Y, and Z are not substituted in the polycyclic compound represented by the above formula (II). This polycyclic compound is an acenaphtho[1,2-b]quinoxaline derivative represented by the following general formula (IV).

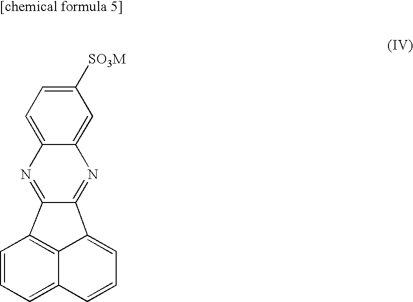

Here, in the formula (IV), the M is the same as the formula (I).

Also, the polycyclic compound can be obtained, for example, by dehydration condensation reaction of an aromatic diamine compound and an acenaphthenequinone derivative.

Specifically, by sulfonation of the aromatic diamine or the substituted aromatic diamine, an aromatic diamine compound in which an —SO₃M group is introduced can be obtained. The sulfonation is performed by using inorganic sulfonic acids such as sulfuric acid, fuming sulfuric acid, chlorosulfonic acid, or the like. Next, by condensation reaction of the aromatic diamine compound in which an —SO₃M group is introduced and an acenaphthoquinone or a substituted acenaphthoquinone, the polycyclic compound having an —SO₃M group can be obtained (refer to the following reaction formula (a)). In the case where the M in the obtained polycyclic compound is not hydrogen, the polycyclic compound having an —SO₃H group can be obtained by bringing the compound into contact with a proper acid as described in the following reaction formula (a).

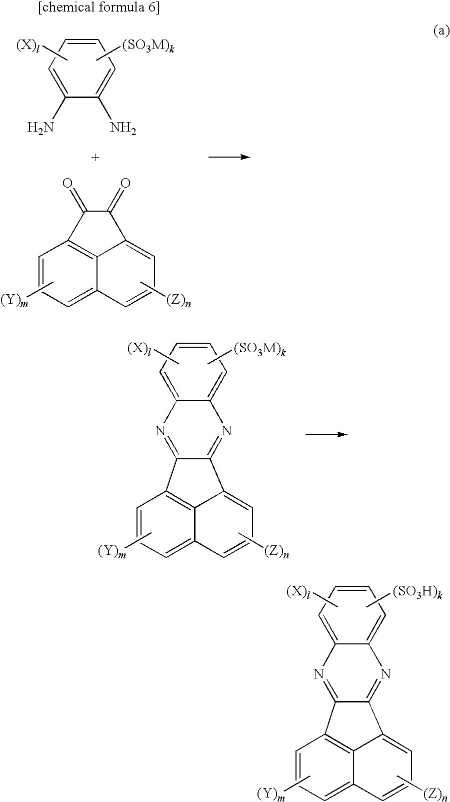

The birefringent film of the present invention can be produced by, for example, dissolving the above polycyclic compound in a proper solvent so as to be in a state of a liquid crystal phase, and coating and drying the solution on the substrate. The coating film produced by coating and drying the solution on the substrate is a birefringent film of the present invention.

The polycyclic compound can form a stable liquid crystal phase in the solution. Therefore, by performing solvent casting method to the solution containing the polycyclic compound, a transparent birefringent film having a high in-plane birefringence index and no or little absorption in the visible light region can be obtained.

In the birefringent film, an arbitrary additive may be added other than the polycyclic compound. Examples of the additive include a plasticizer, a thermal stabilizer, an optical stabilizer, a lubricant, an antioxidant, an ultraviolet absorber, a flame retardant, a coloring agent, an antistatic agent, a compatibility improving agent, a cross-linking agent, and a thickening agent. The additive amount of these additives is larger than 0 part and 10 parts by mass or less with respect to 100 parts by mass of the polycyclic compound.

The birefringent film of the present invention can be formed into film form by casting the solution. Therefore, the present invention can provide a relatively-thin birefringent film.

The thickness of the birefringent film is preferably 0.05 μm or more and more preferably 0.1 μm or more. The upper limit of the thickness of the birefringent film is not particularly limited, and it may be set arbitrary in accordance with an in-plane retardation value and/or a retardation value in the thickness direction. The birefringent film is preferably thin, so that the thickness thereof is preferably 10 μm or less, more preferably 5 μm or less, and particularly preferably 3 μm or less.

Further, the birefringent film has an index ellipsoid satisfying a relationship of nz>nx>ny and a relatively high in-plane birefringence index. Therefore, the birefringent film is thin remarkably as compared to a conventional birefringent film, but has a predetermined retardation value.

A transmittance (T[590]) of the birefringent film at a wavelength of 590 nm is preferably 80% or more and more preferably 90% or more. A Haze value of the birefringent film is preferably 5% or less, more preferably 4% or less, and particularly preferably 3% or less. An image display device including the birefringent film having the Haze value within the above range is excellent in displaying characters. The Haze value is a value measured according to JIS-K7105.

The in-plane birefringence index (Δn_{xy[}590]) of the birefringent film at a wavelength of 590 nm is preferably 0.01 or more, more preferably from 0.05 to 0.3, and particularly preferably from 0.1 to 0.3.

The in-plane retardation value (Re[590]) of the birefringent film at a wavelength of 590 nm is set optionally in accordance with an object. The Re[590] is preferably 10 nm or more, more preferably from 20 nm to 500 nm, and particularly preferably from 50 nm to 300 nm.

The retardation value (Rth[590]) in the thickness direction of the birefringent film at a wavelength of 590 nm is set arbitrary in accordance with an object. The Rth[590] is preferably −10 nm or less, more preferably −500 nm to −10 nm, and particularly preferably −100 nm to −10 nm.

The Nz coefficient of the birefringent film is preferably less than 0, more preferably more than −0.5 and less than 0, and particularly preferably −0.3 to −0.1. The birefringent film having the Nz coefficient within the above range can utilize for optical compensation of liquid crystal displays having various driving modes.

### <Method for Producing Birefringent Film of the Present Invention>

In one embodiment, the birefringent film of the present invention can be obtained by a method including the following steps.

Step (1): A step of preparing a solution exhibiting a liquid crystal phase, containing at least the polycyclic compound and a solvent.

Step (2): A step of preparing a substrate.

Step (3): A step of applying the solution of the above step (1) onto the surface of the substrate of the above step (2) and drying the solution.

In addition, any of the step (1) and the step (2) may be performed first, or both the steps may be performed simultaneously in parallel, and the order of the steps is not limited.

### [Step (1)]

The step (1) is a step of preparing a solution containing at least the polycyclic compound.

The polycyclic compound can be appropriately selected from the compounds exemplified above. The polycyclic compound can be selected from, for example, the polycyclic compounds represented by the general formula (I), which has different number of substitutions and/or different substitution positions of the —SO₃M group, and the polycyclic compounds having different kinds and/or different number of substitutions and/or different substitution positions of X, Y, and Z in the general formula (I). These polycyclic compounds may be used alone or in combination of two or more thereof.

As a solvent, any of solvents in which the above polycyclic compounds can be dissolved to exhibit a liquid crystal phase (preferably a nematic liquid crystal phase) is selected.

The solvent is, for example, an inorganic solvent such as water, or an organic solvent such as alcohols, ketones, ethers, esters, amides, or cellosolves. The organic solvent is, for example, n-butanol, 2-butanol, cyclohexanol, isopropyl alcohol, t-butyl alcohol, glycerin, ethylene glycol, acetone, methyl ethyl ketone, methyl isobutyl ketone, cyclohexanone, cyclopentanone, 2-pentanone, 2-hexanone, tetrahydrofuran, dioxane, ethyl acetate, butyl acetate, methyl lactate, dimethyl formamide, dimethylacetamide, N-methylpyrolidone, methyl cellosolve, or ethyl cellosolve. These solvents can be used alone or in combination of two or more thereof.

The solvent is preferably a water-based solvent and particularly preferably water. Electric conductivity of water is preferably 20 μS/cm or less, more preferably 0.001 μS/cm to 10 μS/cm, and particularly preferably 0.001 μS/cm to 5 μS/cm. The lower limit of the electric conductivity of water is 0 μS/cm. By use of water in which the electric conductivity is within the above range, a birefringent film having an index ellipsoid satisfying a relationship of nz>nx>ny can be obtained.

A concentration of the polycyclic compound in the solution is prepared optionally and appropriately range as far as the solution exhibits a lyotropic liquid crystal phase. The concentration of the polycyclic compound in the solution is preferably 3% to 40% by mass, more preferably 3% to 30% by mass, particularly preferably 5% to 30% by mass, and most preferably 10% to 30% by mass. The solution having the concentration within the above range can exhibit a stable liquid crystalline state.

In the solution, an optional additive may be added appropriately. Examples of the additive include a surfactant, a plasticizer, a thermal stabilizer, an optical stabilizer, a lubricant, an antioxidant, an ultraviolet absorber, a flame retardant, a coloring agent, an antistatic agent, a compatibility improving agent, a cross-linking agent, a thickening agent, and the like. The additive amount of these additives is preferably more than 0 part and 10 or less parts by mass with respect to 100 parts by mass of the solution.

In the solution, a surfactant may be added. The surfactant is added for improving wettability and coatability of the surface of the substrate to the solution containing the polycyclic compound. The surfactant is preferably a nonionic surfactant. The additive amount of the surfactant is preferably more than 0 part and 5 or less parts by mass with respect to 100 parts by mass of the solution.

### [Step (2)]

The step (2) is a step of preparing a substrate. Preferably, the substrate is subjected to hydrophilizing treatment on at least one surface thereof.

The substrate is a member used for uniformly developing the above solution containing the polycyclic compound. The substrate may be selected optionally and appropriately. The substrate is, for example, a glass substrate, a quartz substrate, a polymeric film, a plastic substrate, a metal substrate made of aluminum or iron, a ceramic substrate, a silicon wafer, and the like. The substrate is preferably the glass substrate or the polymeric film.

The glass substrate is not particularly limited, and may be selected appropriately. The glass substrate is preferably a glass plate used for a liquid crystal cell generally. Examples of this glass plate include soda-lime glass (blue sheet) containing an alkaline component, or low-alkali borax acid glass. A commercial glass substrate may be directly used for the glass substrate. Examples of the commercial glass substrate include glass code: 1737 manufactured by Corning Incorporated, glass code: AN635 manufactured by Asahi Glass Co., Ltd., and glass code: NA-35 manufactured by NH Techno Glass Corporation.

A resin forming the polymeric film is not particularly limited. The polymeric film preferably includes a film containing a thermoplastic resin. The thermoplastic resin includes, for example, a polyolefin resin, a cycloolefin-based resin, polyvinyl chloride-based resin, a cellulose-based resin, a styrene-based resin, a polymethylmethacrylate, a polyvinyl acetate, a polyvinylidene chloride-based resin, a polyamide-based resin, a polyacetal-based resin, a polycarbonate-based resin, a polybutylene terephthalate-based resin, a polyethylene terephthalate-based resin, a polysulphone-based resin, a polyether sulphone-based resin, a polyether ether ketone-based resin, a polyarylate-based resin, a polyamide-imide-based resin, a polyimide-based resin, and the like. The thermoplastic resin is used singly or in combination of two kinds or more. The thermoplastic resin may be also used after performing optional and appropriate polymer modification. Examples of the polymer modification include copolymerization, crosslinking, molecular ends, and stereoregularity.

The polymeric film is preferably a film excellent in light transmittance in visible light and transparency. The transmittance (T[590]) of this polymeric film in visible light is preferably 80% or more and more preferably 90% or more. The Haze value of the polymeric film is preferably 3% or less and more preferably 1% or less. Here, the Haze value is a value measured according to JIS-K7105.

In the case where the substrate is the polymeric film, after forming the birefringent film on this substrate, the substrate may be utilized as a protective film.

The substrate is preferably a polymeric film containing a cellulose-based resin. This substrate containing a cellulose-based resin is excellent in wettability to the solution containing the polymeric compound. Therefore, a birefringent film which has small unevenness in thickness can be obtained.

The cellulose-based resin is not particularly limited, and may be selected appropriately. The cellulose-based resin is preferably, for example, a cellulose organic acid ester or a cellulose mixed organic acid ester, in which a part or all of hydroxyl groups of the cellulose are substituted with acetyl groups, propionyl groups and/or butyl groups. Examples of the cellulose organic acid ester include cellulose acetate, cellulose propionate, cellulose butyrate, and the like. Examples of the cellulose mixed organic acid ester include cellulose acetate propionate, cellulose acetate butyrate, and the like. The cellulose-based resin may be obtained by the method described in [0040] and [0041] of Japanese Unexamined Patent Publication No. 2001-188128, for example.

A commercial polymeric film may be directly used for the substrate. Alternatively, a commercial polymeric film for which secondary elaborations are performed may be also used. Examples of this secondary elaboration include a drawing treatment and/or a contraction treatment. Examples of the commercial polymeric film containing a cellulose-based resin include FUJITAC series (trade name: ZRF80S, TD80UF, and TDY-80UL) manufactured by Fuji Photo Film Co., Ltd. and trade name “KC8UX2M” manufactured by Konica Minolta Opt, Inc.

The thickness of the substrate is preferably 20 μm to 100 μm. The substrate having a thickness within the above range is excellent in handling ability, and the solvent can be coated on the substrate better.

Further, the hydrophilizing treatment is subjected to one surface of the substrate. The hydrophilizing treatment is a treatment for decreasing a contact angle of the substrate to water. The hydrophilizing treatment is performed for improving wettability and coatability of the surface of the substrate to the solution containing the polycyclic compound.

The hydrophilizing treatment is a treatment for decreasing a contact angle of the substrate to water at a temperature of 23° C. preferably 10% or more, more preferably 15% to 80%, and particularly preferably 20% to 70% as compared with a state before the treatment. This decreasing ratio (%) is calculated from the expression: {(contact angle before treatment−contact angle after treatment)/contact angle before treatment}×100.

The hydrophilizing treatment is a treatment for decreasing the contact angle of the substrate to water at a temperature of 23° C. preferably 5° or more, more preferably 10° to 65°, and particularly preferably 20° to 60° as compared with a state before the treatment.

In addition, the above hydrophilizing treatment is a treatment for setting the contact angle of water on the substrate at a temperature of 23° C. preferably 5° to 60°, more preferably 5° to 50°, and particularly preferably 5° to 45°. By using a substrate, in which the contact angle is within the above range, a birefringent film having small unevenness in thickness can be obtained.

The hydrophilizing treatment can be any optional and appropriate method. For example, the hydrophilizing treatment may be a dry treatment or a wet treatment. Examples of the dry treatment include a discharge treatment such as a corona treatment, a plasma treatment, or a glow discharge treatment; a flame treatment; an ozone treatment; an UV ozone treatment; an ionization active ray treatment such as an ultraviolet treatment or an electron beam treatment; and the like. Examples of the wet treatment include an ultrasonic treatment using a solvent such as water or acetone; an alkali treatment; an anchor coat treatment; and the like. The treatment can be used singly or in combination of two kinds or more.

The hydrophilizing treatment is preferably the corona treatment, the plasma treatment, the alkali treatment, or the anchor coat treatment. The use of the substrate, which is subjected to these treatments, allows a birefringent film having a high alignment and small unevenness in thickness to be obtained. With regard to the condition of the hydrophilizing treatment (for example, treating time or intensity), it can be set to be a suitable and appropriate value as far as the contact angle of water on the substrate is within the above range.

The corona treatment is typically a treatment for modifying the surface of the substrate by passing the substrate through corona discharge. The corona discharge is caused in such a manner that air between the electrodes is subjected to dielectric breakdown and ionized by impressing high frequency and high voltage between a grounded dielectric roll and an insulated electrode. The plasma treatment is typically a treatment for modifying the surface of the substrate by passing the substrate through low-temperature plasma. The low-temperature plasma is caused in such a manner that glow discharge is caused in inorganic gases such as low-pressure inert gas, oxygen gas and halogen gas, and then a part of the gaseous molecules are ionized. The ultrasonic treatment is typically a treatment for removing contaminations on the surface of the substrate and improving wettability thereof. The alkali treatment is typically a treatment for modifying the surface of the substrate by immersing the substrate in an alkali treatment solution such that a basic material is dissolved in water or an organic solvent. The anchor coat treatment is typically a treatment for coating an anchor coat agent on the surface of the substrate.

### [Step (3)]

The step (3) is a step of applying the solution prepared in the above step (1) onto the surface (a hydrophilized surface if the surface of the substrate has been subjected to a hydrophilizing treatment) of the substrate prepared in the above step (2) and drying the solution.

The application rate of the solution is not particularly limited, but it is preferably 10 mm/second or more, more preferably 50 mm/second or more, and particularly preferably 100 mm/second or more. An upper limit of the application rate is preferably 8000 mm/second, more preferably 6000 mm/second, and particularly preferably 4000 mm/second. By setting the application rate in the above range, a shear force suitable for orienting a polycyclic compound is applied to the solution. Therefore, a birefringent film having an index ellipsoid satisfying a relationship of nz>nx>ny and having small unevenness in thickness can be obtained.

With regard to a method of coating the solution on the substrate, a coating method using an appropriate and proper coater may be used. Examples of the coater include a reverse roll coater, a positive rotation roll coater, a gravure roll coater, a knife coater, a rod coater, a slot die coater, a slot orifice coater, a curtain coater, a fountain coater, an air doctor coater, a kiss coater, a dip coater, a bead coater, a blade coater, a cast coater, a spray coater, a spin coater, an extrusion coater, a hot-melt coater, and the like. The coater is preferably the reverse roll coater, the positive rotation roll coater, the gravure roll coater, the rod coater, the slot die coater, the slot orifice coater, the curtain coater, and the fountain coater. By using these coaters for coating the solution, a birefringent film having small unevenness in thickness may be obtained.

With regard to a method of drying the solution, an optional and appropriate method may be used. Examples of the drying method include an air-circulation thermostat oven in which hot air or cold air is circulated; a heater using a microwave, a far infrared ray, or the like; a roll, a heat pipe roll, or a metal belt heated for temperature regulation; or the like.

The temperature for drying the solution is below or equal to the isotropic phase transition temperature of the solution, and the temperature is preferably raised gradually from a low temperature to a high temperature. The above drying temperature is preferably 10° C. to 80° C., and more preferably 20° C. to 60° C. Within such a temperature range, a birefringent film having small unevenness in thickness can be obtained.

The period of time for drying the solution can be selected optionally and appropriately depending on the drying temperature and the kind of the solvent. In order to obtain a birefringent film having small unevenness in thickness, the drying time is, for example, 1 minute to 30 minutes and preferably 1 minute to 10 minutes.

### [Other Step]

A production method of the birefringent film of the present invention preferably comprises a step (4) in addition after the above step (1) to (3).

Step (4): a step of bringing the birefringent film obtained in the above step (3) into contact with a solution containing at least one kind of a compound salt selected from the group consisting of aluminum salt, barium salt, lead salt, chromium salt, strontium salt, and compound salts having two or more amino groups within a molecule.

In the present invention, the step (4) is performed for imparting insoluble property or hardly-soluble property in water to the obtained birefringent film. Examples of the compound salt include aluminum chloride, barium chloride, lead chloride, chromium chloride, strontium chloride, 4,4′-tetramethyldiaminodiphenylmethane hydrochloride, 2,2′-dipyridyl hydrochloride, 4,4′-dipyridyl hydrochloride, melamine hydrochloride, tetraminopyrimidine hydrochloride, and the like. These compound salts allow a birefringent film excellent in water resistance to be obtained.

In the solution containing the above compound salt, the concentration of the compound salt is preferably 3% to 40% by mass, and particularly preferably 5% to 30% by mass. By bringing the birefringent film into contact with the solution containing the compound salt in the above range, a birefringent film excellent in water resistance can be obtained.

As a method of bringing the birefringent film obtained in the above step (3) into contact with the solution containing the above compound salt, optional method can be used. Examples of the method include a method of coating the solution containing the above compound salt onto the surface of the birefringent film, a method of immersing the birefringent film into the solution containing the above compound salt, or the like. In the case where these methods are used, an obtained birefringent film is preferably washed with water or an optional solvent. In addition, a laminated film excellent in adhesion properties of the interface between the substrate and the birefringent film may be obtained by drying the birefringent film after washing.

### <Use of Birefringent Film of the Present Invention>

Use of a birefringent film of the present invention is not particularly limited, but the film is used for an optical member of a liquid crystal display representatively. Examples of this optical member include a λ/4 plate, a λ/2 plate, a view angle widening film, and an antireflection film for flat panel displays.

In one embodiment of the present invention, a laminated film is provided by laminating an other film on the birefringent film of the present invention.

In other embodiment of the present invention, a polarizing plate may be provided by laminating a polarizer on the birefringent film of the present invention.

The polarizing plate is a laminated film comprising at least the birefringent film of the present invention and a polarizer. The polarizing plate may be laminated the substrate or the other optical film. Examples of the other optical film include a birefringent film other than the birefringent film of the present invention, an optional protective film, or the like. Practically, an appropriate adhesive layer is provided between each of layers constituting the polarizing plate, and each of layers are adhered.

An angle at which the polarizer is adhered to the birefringent film in the above-mentioned polarizing plate can be appropriately determined in accordance with an object. When the polarizing plate is used, for example, as an antireflection film, the polarizer is adhered to the birefringent film such that the angle which is formed by the absorption axis direction of the above polarizer and the slow axis direction of the birefringent film is preferably 25 to 65° and more preferably 35 to 55°. Further when the polarizing plate is used, for example, as a view angle widening film, the polarizer is adhered to the birefringent film such that the absorption axis direction of the polarizer is substantially parallel to or substantially orthogonal to the slow axis direction of the birefringent film. Here, “substantially parallel” includes a case where an angle formed by the absorption axis direction of the polarizer and the slow axis direction of the birefringent film is 0°±10° and preferably 0°±5°. Also, “substantially orthogonal” includes a case where an angle formed by absorption axis direction of the polarizer and the slow axis direction of the birefringent film is 90°±10° and preferably 90°±5°.

The polarizer is an optical film having the property of converting natural light or polarized light into linearly polarized light. The polarizer is, for example, a drawn film having polyvinyl alcohol resin containing iodine or dichromatic dye as a main component. In general, a thickness of the polarizer is 5 μm to 50 μm.

The adhesive layer can be selected from any optional one as far as the adhesive layer joins planes of adjacent members, which are integrated by practically sufficient adhesive force and adhesive time. Examples of a material for forming the adhesive layer include an adhesive agent, a pressure-sensitive agent, and an anchor coat agent. The adhesive layer may be a multi-layered structure such that an anchor coat agent layer is formed on the surface of an adherend to form an adhesive agent layer or a pressure-sensitive agent layer thereon, or a thin layer unrecognizable with the naked eye (also referred to as hairline). The adhesive layer disposed on one side of the polarizer and the adhesive layer arranged on the other side thereof may be the same or different.

The birefringent film of the present invention and a laminated film containing the birefringent film are used by being mounted on various image display devices.

The image display devices include an organic EL display, a plasma display other than a liquid crystal display. A preferable use of the image displays is a television set, and particularly preferably a large-scale television set having a screen size of 40 inches or more. In the case where the image display device is a liquid crystal display, preferable use thereof is OA apparatus such as a television set, a personal computer monitor, a notebook personal computer, and a copying machine; portable apparatus such as a portable telephone, a watch, a digital camera, a portable digital assistance (PDA), and a portable game machine; a home-use electric apparatus such as a video camera and an electronic range; apparatus to be mounted on a vehicle such as a back monitor, a monitor for a car navigation system, and a car audio device; an exhibition apparatus such as an information monitor for commercial shops; guarding apparatus such as a monitor for supervision; and assisting and medical apparatus such as a monitor for assisting senior persons and a monitor for medical use.

### EXAMPLES

Hereinafter, the present invention will be further described by showing Examples. However, the present invention is not limited to following Examples.

Here, measuring methods used in Examples are as follows.

### (1) Measuring Method of Thickness:

The thickness was measured by peeling off a part of a birefringent film formed on the surface of a substrate (glass plate), and measuring a difference in level between the substrate and the birefringent film with a stylus profilometry manufactured by ULVAC, Inc. (trade name: “DEKTAK”).

### (2) Measuring Method of T[590], nx, ny, nz, Re[590], Rth[590], and Nz Coefficient:

The T[590] and the like were measured by using an apparatus manufactured by Oji Scientific Instruments, trade name: “KOBRA21-ADH”, at 23° C. Average refractive indexes were measured by using an Abbe refractometer (manufactured by ATAGO Co., Ltd., trade name: “DR-M4”).

(3) Measuring Method of Δn_{xy[}590]:

The Δn_{xy[}590] was calculated by Rth[590]/thickness (nm).

### (4) Method for Identifying Liquid Crystal Phase:

A solution was sandwiched between two slide glasses and placed on a hot stage (manufactured by Mettler-Toledo International Inc., trade name: “FP28HT”), and the solution was observed with a polarizing microscope (manufactured by Olympus Corporation, trade name: “BX 50”) while varying a temperature of the solution to identify a liquid crystal phase.

### Synthesis Example 1

### Synthesis of 3,4-diaminobenzenesulfonic acid

60 ml of 4% fuming sulfuric acid was charged into a reaction vessel equipped with a stirrer, and 10.0 g of 1,2-phenylenediamine was gradually added while stirring the content in the vessel, and the resulting mixture was heated to 140° C. and reacted for 4 hours. The resulting solution was cooled and 92.5 ml of ion exchange water was added while maintaining a temperature of the solution at 60° C. or less to dilute the solution, and the diluted solution was cooled to 25° C. or less and then stirred for 3 hours. A precipitate was separated by filtration and a filtrate was recrystallized with ion exchange water twice, and a solid substance after filtration was vacuum dried for 12 hours to obtain 13.7 g of 3,4-diaminobenzenesulfonic acid.

### Synthesis Example 2

### Synthesis of acenaphtho[1,2-b]quinoxaline-9-sulfonic acid

60 ml of isopropanol and 57 ml of ion exchange water were charged into a reaction vessel equipped with a stirrer, and 4.0 ml of a 50% aqueous solution of sodium hydroxide was added while bubbling with nitrogen gas and stirring the content in the vessel. To the resulting solution, 9.5 g of 3,4-diaminobenzenesulfonic acid obtained in [Synthesis Example 1] was added to dissolve the mixture completely. 9.2 g of acenaphthenequinone was added over 1 hour while maintaining the solution at a temperature of 40 to 45° C., and the resulting mixture was reacted for 1 hour while maintaining the mixture at a temperature of 35 to 40° C. (see the following reaction formula (b)). 300 ml of isopropanol was added into another reaction vessel equipped with a stirrer and heated to 75° C. and stirred, and the aforementioned reacted solution was added thereto. Thereafter, by keeping on stirring the resulting mixture for 1 hour while maintaining the mixture at a temperature of 70 to 75° C., a sodium salt of acenaphtho[1,2-b]quinoxaline-9-sulfonic acid was precipitated to obtain a raw product by filtration. The raw product was dispersed in 210 ml of acetic acid, and 2.8 ml of sulfuric acid was added thereto, and the resulting mixture was stirred for 30 minutes. Thereafter, the resulting mixture was cooled to room temperature to precipitate acenaphtho[1,2-b]quinoxaline-9-sulfonic acid and the precipitate was separated by filtration to obtain a raw product. The raw product was washed with acetic acid to obtain 15 g of acenaphtho[1,2-b]quinoxaline-9-sulfonic acid.

10 g of this product was dissolved in 1 l of ion exchange water (electric conductivity: 0.1 μS/cm), and a 5% aqueous solution of sodium hydroxide was added thereto to prepare an aqueous solution of pH 3.5. The obtained aqueous solution was put in a supply tank, and circulated and filtered while adding reverse osmosis water so as to have a constant liquid rate using a test apparatus equipped with a reverse osmosis membrane manufactured by Nitto Denko Corporation (trade name: “NTR-7430 Filter”). With this circulation/filtration, residual acids were removed until the electric conductivity of waste liquid reached 146 μS/cm to obtain an aqueous solution containing acenaphtho[1,2-b]quinoxaline-9-sulfonic acid.

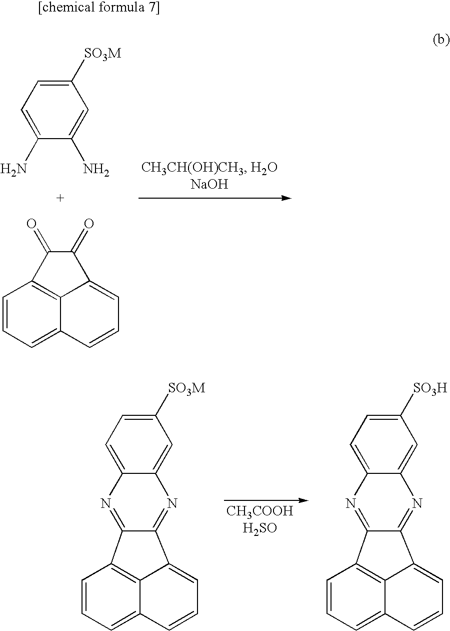

### Example

A 5% aqueous solution of sodium hydroxide was added to the aqueous solution containing acenaphtho[1,2-b]quinoxaline-9-sulfonic acid obtained in Synthesis Example 2 and a pH of the resulting mixture was adjusted to 7. Next, the aforementioned aqueous solution was condensed until the concentration of the acenaphtho[1,2-b]quinoxaline-9-sulfonic acid in the aqueous solution reached 28% by mass using a rotary evaporator. Thereafter, ion exchange water was added to the condensed aqueous solution until the aforementioned concentration reached 24% by mass, and the resulting mixture was stirred. The aqueous solution having such a concentration was observed with a polarizing microscope, and consequently the aqueous solution exhibited a nematic liquid crystal phase at 23° C.

The aqueous solution was applied (wet thickness: 3 μm) onto a 1.3 mm thick glass substrate using a bar coater (manufactured by BUSCHMAN Corporation, trade name “mayer rot HS1.5”), and naturally dried in a thermostat room having a temperature of 23° C. As a result, a birefringent film in which an index ellipsoid satisfies a relationship of nz>nx>ny was obtained on the surface of the glass substrate.

This birefringent film had a thickness of 0.7 μm and the following characteristics.

Δn_{xy[}590]=0.19

T[590]=90%

Re[590]=125 nm

Rth[590]=−19 nm

Nz coefficient=−0.16

1. A birefringent film comprising a lyotropic liquid crystalline polycyclic compound having an —SO₃M group, wherein M represents a counterion, and having an index ellipsoid satisfying a relationship of nz>nx>ny.

2. The birefringent film according to claim 1, wherein the polycyclic compound is an acenaphtho[1,2-b]quinoxaline derivative represented by the following general formula (I): 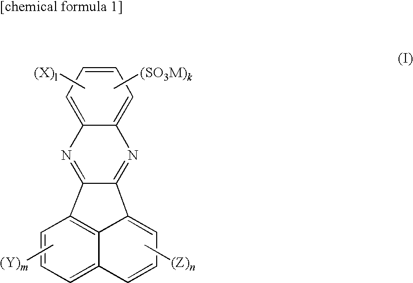
wherein X, Y, and Z each independently represent a halogen atom, an alkoxy group, an —R group, an —OM group, a —COOM group, an —OCOR group, an —NHCOR group, a —CONHR group, an —NH₂ group, an —NO₂ group, a —CF₃ group, a —CN group, an —OCN group, an —SCN group, an —SM group, or a —PO₃M group; M represents a counterion; R represents hydrocarbon; k, l, m, and n represent the number of substitutions, k is an integer of 1 to 4, and l, m, and n are each independently an integer of 0 to 3.

3. The birefringent film according to claim 1, wherein the polycyclic compound contains at least one of acenaphtho[1,2-b]quinoxaline derivatives represented by the following general formulae (II) and (III): 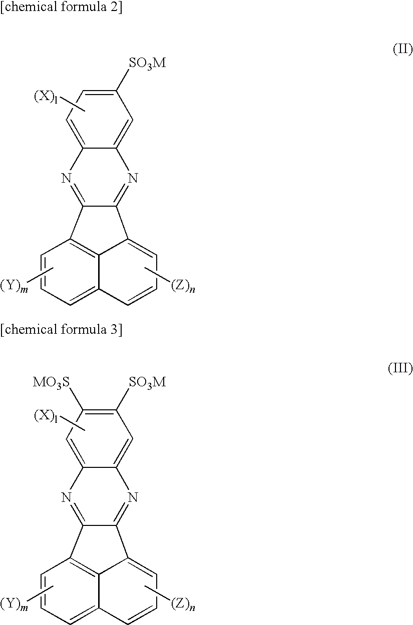
wherein X, Y, and Z each independently represent a halogen atom, an alkoxy group, an —R group, an —OM group, a —COOM group, an —OCOR group, an —NHCOR group, a —CONHR group, an —NH₂ group, an —NO₂ group, a —CF₃ group, a —CN group, an —OCN group, an —SCN group, an —SM group, or a —PO₃M group; M represents a counterion; R represents hydrocarbon; l, m, and n represent the number of substitutions, and m and n are each independently an integer of 0 to 3, and in the formula (II), l is an integer of 0 to 3, and in the formula (III), l is an integer of 0 to 2.

4. The birefringent film according to claim 1, wherein an in-plane retardation value (Re[590]) at a wavelength of 590 nm is 10 nm or more.

5. The birefringent film according to claim 1, wherein a retardation value (Rth[590]) in the thickness direction at a wavelength of 590 nm is −10 nm or less.

6. The birefringent film according to claim 1, having an Nz coefficient of more than −0.5 and less than 0.

7. The birefringent film according to claim 1, which is obtained by applying a solution containing the polycyclic compound onto a substrate and drying the solution.

8. A laminated film having the birefringent film according to claim 1 and another film.

9. The laminated film according to claim 8, wherein the another film includes a polarizer.

10. An image display device comprising the birefringent film according to claim 1.
